(19) United States
(12) Patent Application Publication
(10) Pub. No.: US 2021/0093412 A1
(43) Pub. Date: Apr. 1, 2021

(54) MEDICAL DEVICE WITH ILLUMINATOR

(71) Applicant: Auris Health, Inc., Redwood City, CA (US)

(72) Inventor: Steven E. Yampolsky, Oakland, CA (US)

(21) Appl. No.: 17/037,475

(22) Filed: Sep. 29, 2020

Related U.S. Application Data
(60) Provisional application No. 62/908,437, filed on Sep. 30, 2019.

(51) Int. Cl. A61B 90/30; A61B 90/00; A61B 34/37

(52) U.S. Cl. CPC A61B 90/30; A61B 90/36; A61B 2034/301; A61B 2090/306; A61B 34/37

(57) ABSTRACT

Certain aspects relate to medical instrument systems having illuminators. Such a system can include a medical instrument and a robotic arm. The medical instrument can include an elongate shaft having a distal end, an optical fiber extending along the elongate shaft, and a wavelength conversion medium disposed at the distal end of the elongate shaft. The wavelength conversion medium can be configured to receive light having a first wavelength profile from the optical fiber and emit light having a second wavelength profile different from the first wavelength profile. The robotic arm can be configured to manipulate the medical instrument. The robotic arm can be articulable to position the medical instrument with respect to a patient. The robotic arm can be configured to actuate the elongate shaft to move the wavelength conversion medium within the patient.

### CROSS-REFERENCE TO RELATED APPLICATION(S)

This application claims the benefit of U.S. Provisional Application No. 62/908,437, filed Sep. 30, 2019, which is hereby incorporated by reference in its entirety.

### TECHNICAL FIELD

The systems and methods disclosed herein are directed to medical instrument systems, and more particularly to medical instruments that include a light source for illuminating a target anatomy.

### BACKGROUND

Medical procedures, such as endoscopy, laparoscopy, etc., may involve accessing and visualizing the inside of a patient's anatomy for diagnostic and/or therapeutic purposes. For example, gastroenterology, urology, and bronchology involve medical procedures that allow a physician to examine patient lumens, such as the ureter, gastrointestinal tract, and airways (bronchi and bronchioles). During these procedures, a thin, flexible tubular tool or instrument, known as an endoscope, is inserted into the patient through an orifice (such as a natural orifice) and advanced towards a tissue site identified for subsequent diagnosis and/or treatment. The medical instrument may need to illuminate anatomy of the patient, but needs still exist for illuminating the patient anatomy.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopic procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopic procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 shows an example robotic medical system, according to some embodiments.

FIG. 22 shows an example configuration of a robotic medical system where the light source is disposed outside of the medical instrument.

FIG. 23 shows an example robotic medical system where an elongate shaft is configured to translate axially through an instrument base.

FIG. 24 shows an example configuration of an distal illumination component.

FIG. 25 shows an end view of a distal end of an elongate shaft of an example medical instrument.

FIG. 26 shows an example method for illuminating a target using a robotic medical system.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

A. Robotic System—Cart.

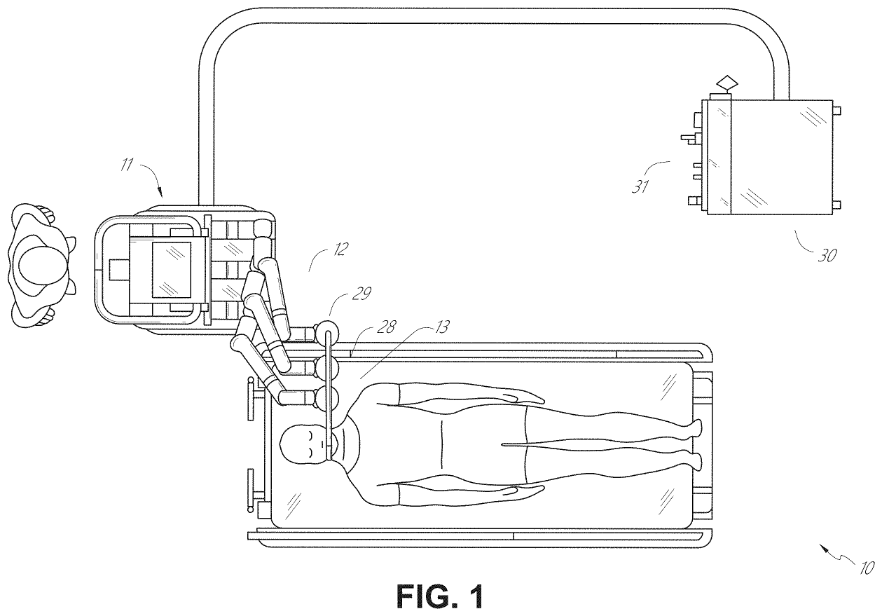

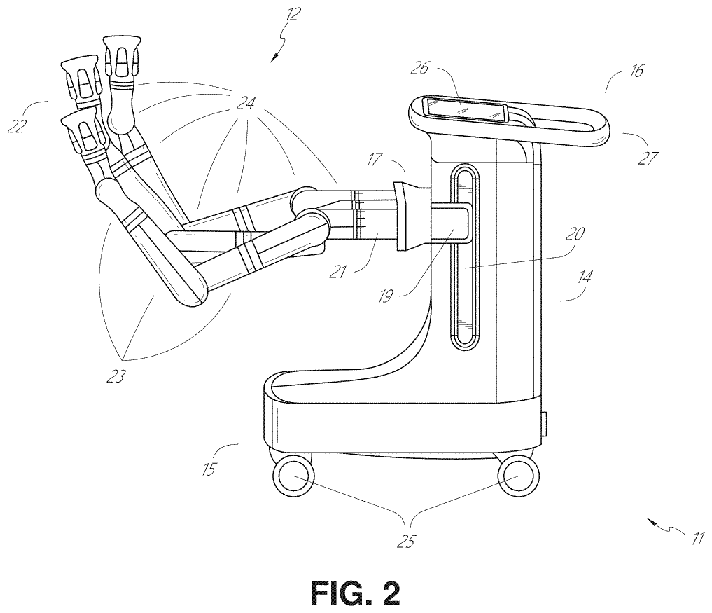

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independently of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart 11, the support for controls, optics, fluidics, and/or navigation may be provided through a separate cable.

FIG. 2 provides a detailed illustration of an embodiment of the cart 11 from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when the carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Having redundant degrees of freedom allows the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intraoperative data. Potential preoperative data on the touchscreen 26 may include preoperative plans, navigation and mapping data derived from preoperative computerized tomography (CT) scans, and/or notes from preoperative patient interviews. Intraoperative data on display may include optical information provided from the tool, sensor and coordinate information from sensors, as well as vital patient statistics, such as respiration, heart rate, and/or pulse. The console 16 may be positioned and tilted to allow a physician to access the console 16 from the side of the column 14 opposite the carriage 17. From this position, the physician may view the console 16, robotic arms 12, and patient while operating the console 16 from behind the cart 11. As shown, the console 16 also includes a handle 27 to assist with maneuvering and stabilizing the cart 11.

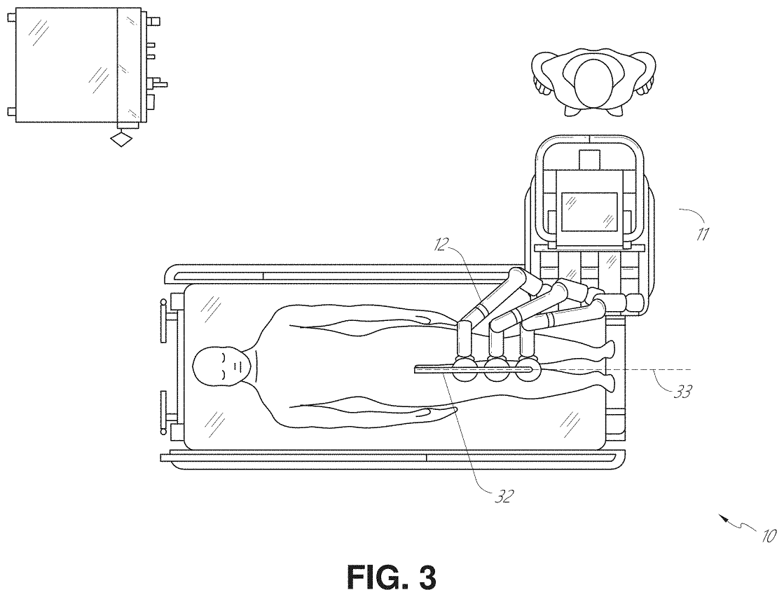

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

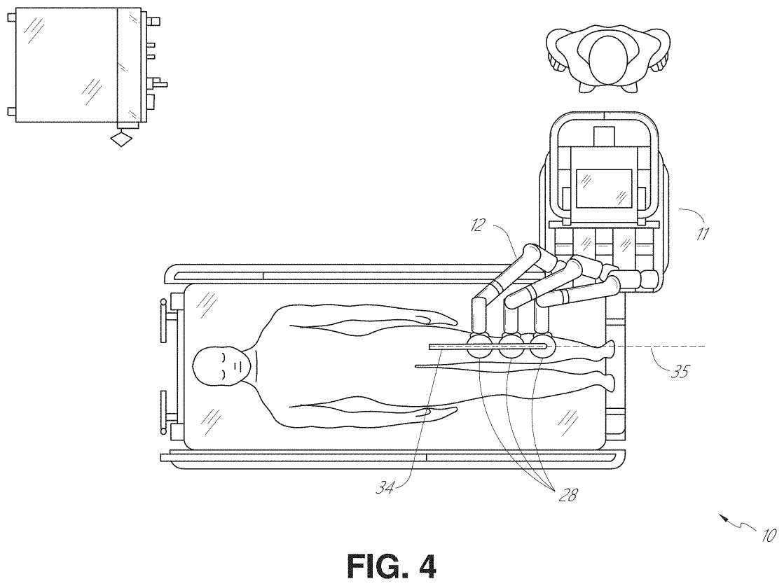

FIG. 4 illustrates an embodiment of a robotically-enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

B. Robotic System—Table.

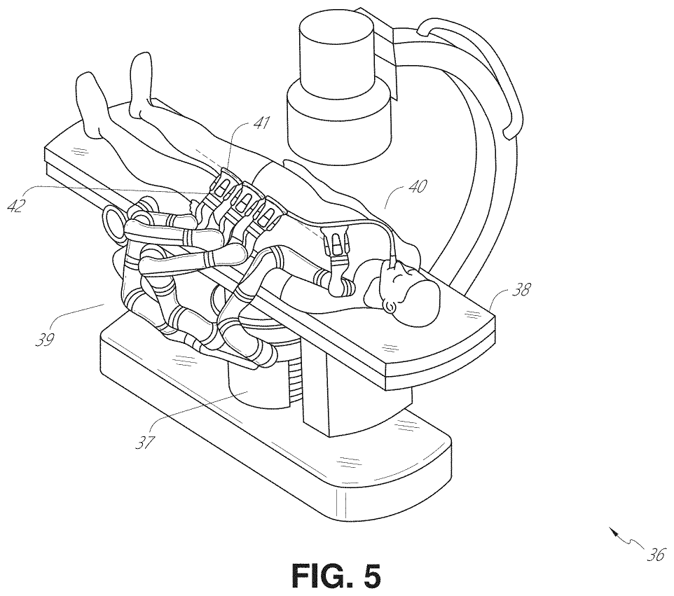

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopic procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

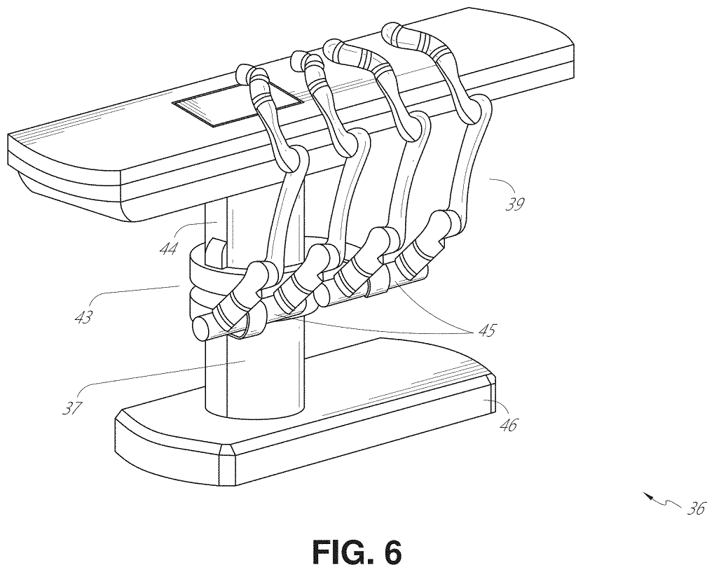

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independently of the other carriages. While the carriages 43 need not surround the column 37 or even be circular, the ring-shape as shown facilitates rotation of the carriages 43 around the column 37 while maintaining structural balance. Rotation and translation of the carriages 43 allows the system 36 to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

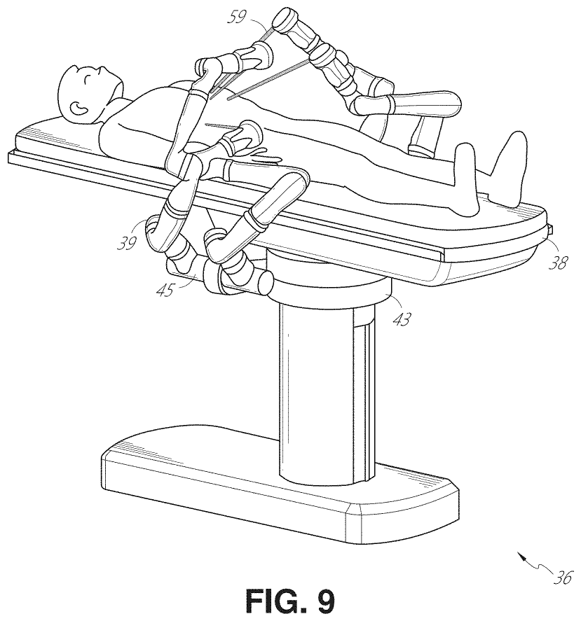

The robotic arms 39 may be mounted on the carriages 43 through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of the table 38 (as shown in FIG. 6), on opposite sides of the table 38 (as shown in FIG. 9), or on adjacent sides of the table 38 (not shown).

The column 37 structurally provides support for the table 38, and a path for vertical translation of the carriages 43. Internally, the column 37 may be equipped with lead screws for guiding vertical translation of the carriages, and motors to mechanize the translation of the carriages 43 based the lead screws. The column 37 may also convey power and control signals to the carriages 43 and the robotic arms 39 mounted thereon.

The table base 46 serves a similar function as the cart base 15 in the cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

With continued reference to FIG. 6, the system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 46 for potential stowage of the robotic arms 39. The tower may also include a master controller or console that provides both a user interface for user input, such as keyboard and/or pendant, as well as a display screen (or touchscreen) for preoperative and intraoperative information, such as real-time imaging, navigation, and tracking information. In some embodiments, the tower may also contain holders for gas tanks to be used for insufflation.

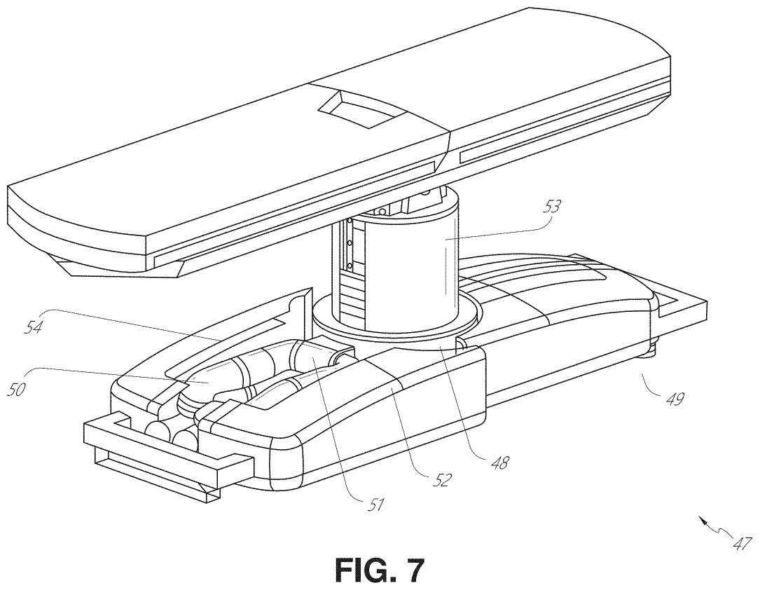

In some embodiments, a table base may stow and store the robotic arms when not in use. FIG. 7 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and robotic arms 50 around column 53, and closed to stow to protect them when not in use. The base covers 52 may be sealed with a membrane 54 along the edges of its opening to prevent dirt and fluid ingress when closed.

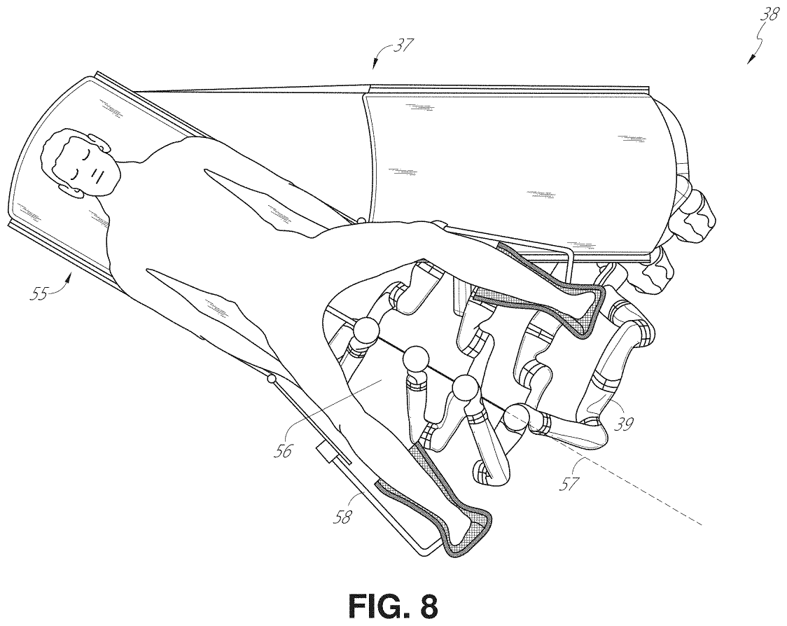

FIG. 8 illustrates an embodiment of a robotically-enabled table-based system configured for a ureteroscopic procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

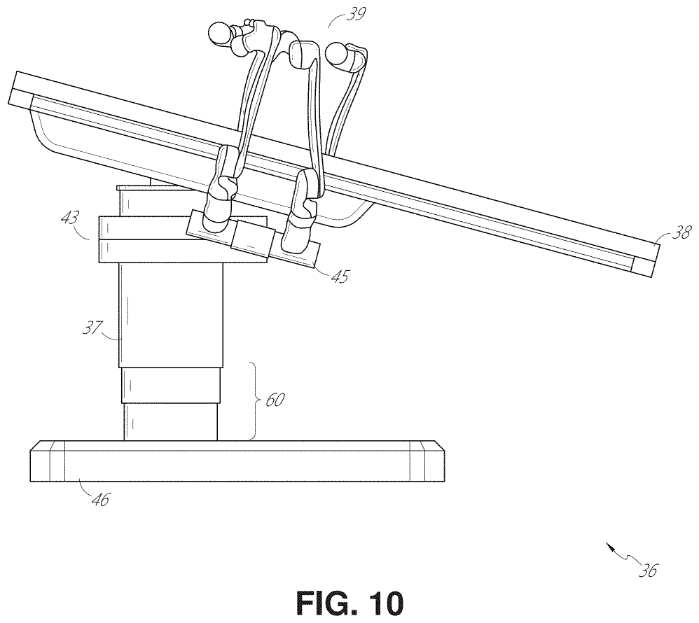

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the robotic arms 39 maintain the same planar relationship with the table 38. To accommodate steeper angles, the column 37 may also include telescoping portions 60 that allow vertical extension of the column 37 to keep the table 38 from touching the floor or colliding with the table base 46.

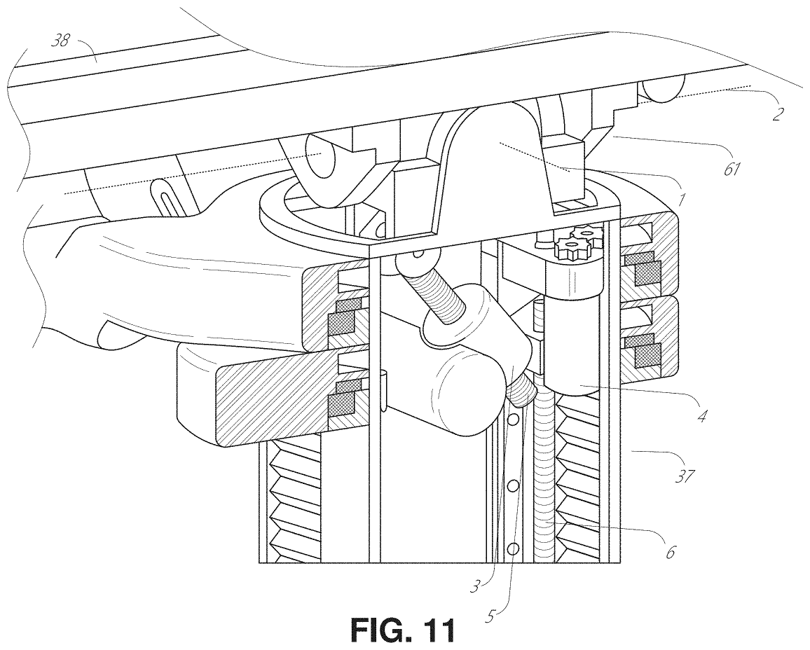

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's upper abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

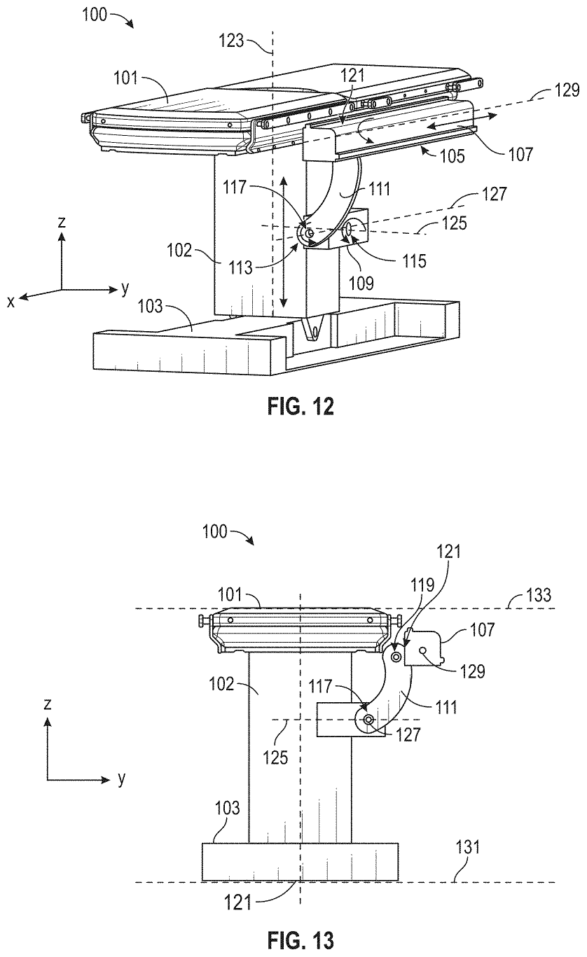

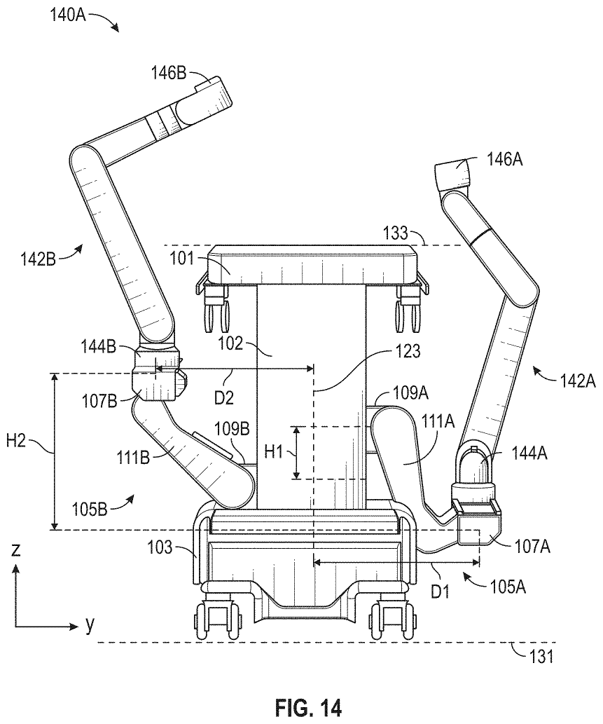

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

C. Instrument Driver & Interface.

The end effectors of the system's robotic arms may comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

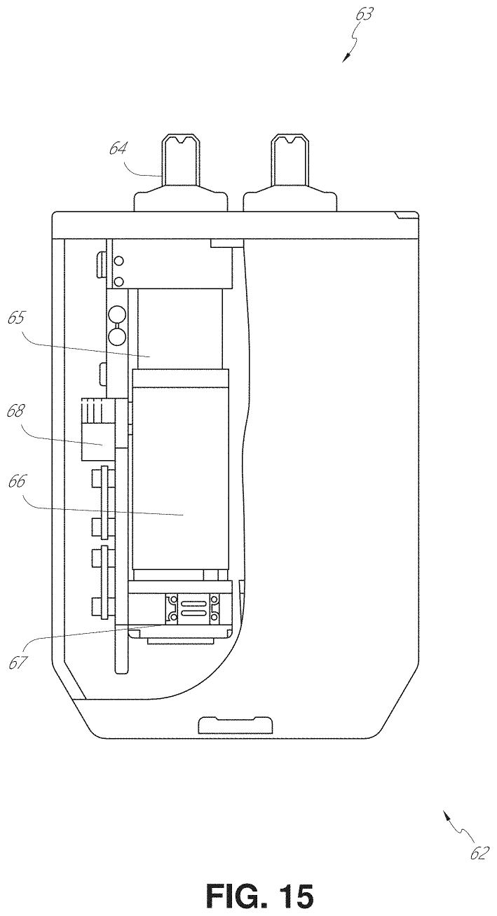

FIG. 15 illustrates an example instrument driver. Positioned at the distal end of a robotic arm, instrument driver 62 comprises one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independently controlled and motorized, the instrument driver 62 may provide multiple (e.g., four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

D. Medical Instrument.

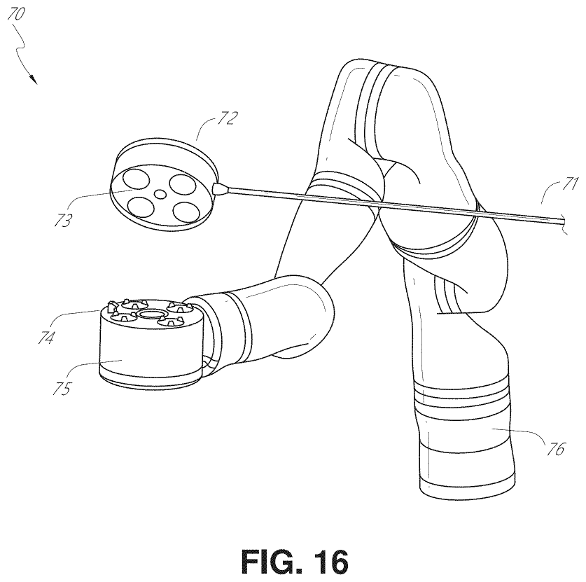

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of the instrument base 72 may share axes of rotation with the drive outputs 74 in the instrument driver 75 to allow the transfer of torque from the drive outputs 74 to the drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the elongated shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at the distal end of the elongated shaft 71, where tension from the tendon causes the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on the drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft 71 may comprise a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include an optical camera. The shaft 71 may also accommodate optical fibers to carry light from proximally-located light sources, such as light emitting diodes, to the distal end of the shaft 71.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft 71. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft 71 during an endoscopic procedure.

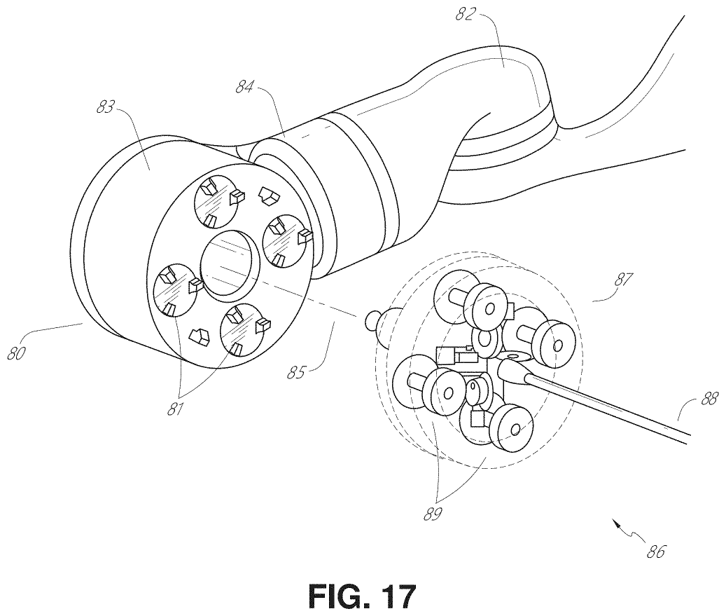

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver 80. Power and controls signals may be communicated from the non-rotational portion 84 of the instrument driver 80 to the rotational assembly 83 through electrical contacts that may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, the instrument shaft 88 extends from the center of the instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

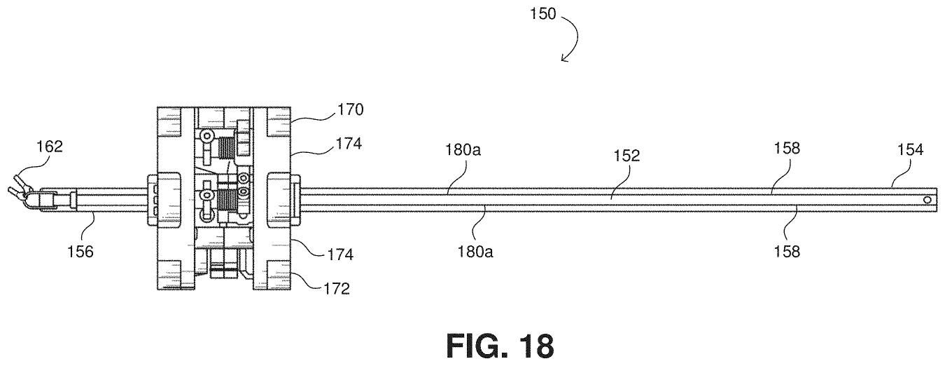

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

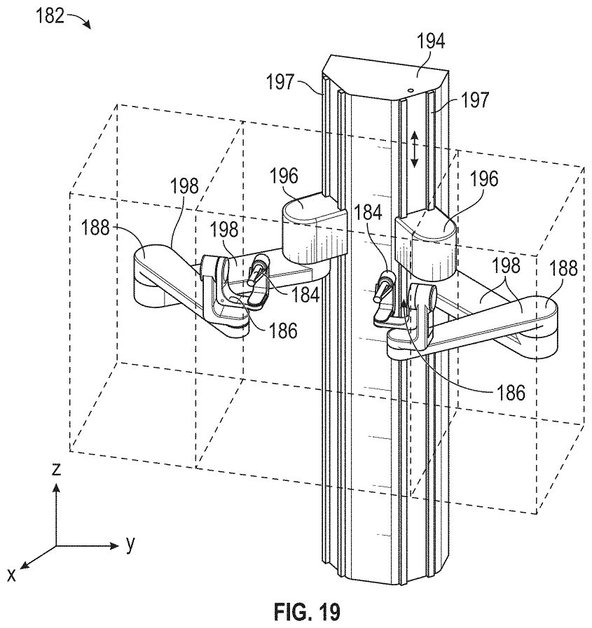

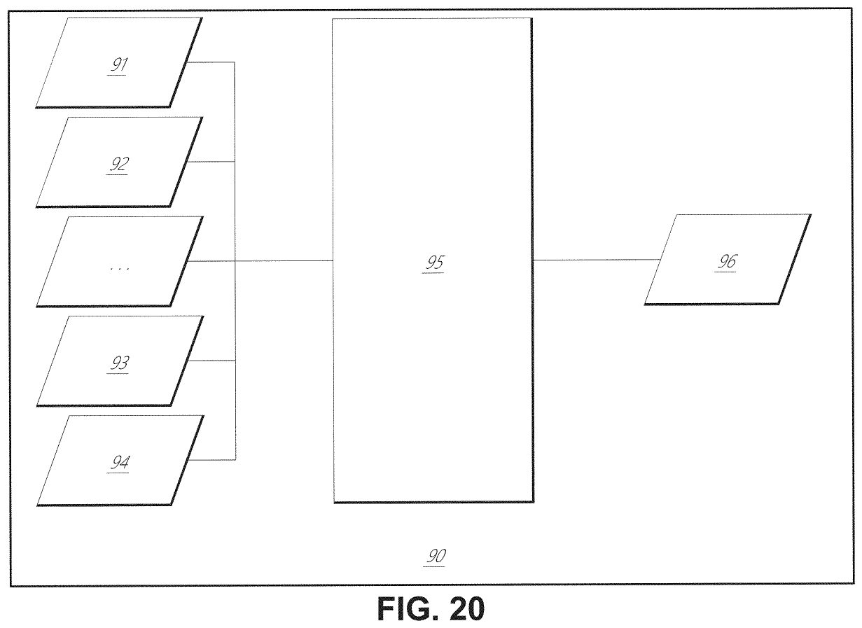

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as preoperative mapping, computer vision, real-time EM tracking, and robot command data may be used individually or in combination to achieve a radiation-free operating environment. In other cases, where radiation-based imaging modalities are still used, the preoperative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart 11 shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Preoperative mapping may be accomplished through the use of the collection of low dose CT scans. Preoperative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data (or image data) 92. The localization module 95 may process the vision data 92 to enable one or more vision-based (or image-based) location tracking modules or features. For example, the preoperative model data 91 may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intraoperatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intraoperatively “registered” to the patient anatomy (e.g., the preoperative model) in order to determine the geometric transformation that aligns a single location in the coordinate system with a position in the preoperative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during preoperative calibration. Intraoperatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Medical Instruments with Illuminators

Embodiments of the disclosure relate to devices, systems, and techniques for medical instruments with one or more illuminators. Illuminators may include light sources, such as light emitting diodes (LEDs), lasers, filaments, and/or other light emitting elements. Illuminators may emit light via radiation, luminescence (e.g., fluorescence, electroluminescence, phosphorescence), etc. The illuminator can include a light source that emits light having a particular wavelength profile (e.g., blue). The medical instruments with illuminator(s) can be used, in some embodiments, with robotically-enabled medical systems, such as those described above with reference to FIGS. 1-20. Examples of medical instruments can include an endoscope, a camera (e.g., with an optical fiber), a basketing tool, a blade tool, a laser tool (e.g., with an optical fiber), and/or other instruments described herein. Example illumination architectures include coupling a light source emitting a first wavelength profile to a wavelength conversion medium that emits light at a second wavelength profile. A medical instrument can include an elongate shaft having a distal end, an optical fiber extending along the elongate shaft, and a wavelength conversion medium. The wavelength conversion medium can be disposed at the distal end of the elongate shaft and/or can be configured to receive light having a first wavelength profile from the optical fiber and emit light having a second wavelength profile different from the first wavelength profile. The medical instrument may be coupled to a robotic arm that is configured to manipulate the medical instrument. The robotic arm can be articulable to position the medical instrument with respect to a patient and/or can be configured to actuate the elongate shaft to move the wavelength conversion medium within the patient.

In some embodiments, the medical instruments can be configured for endoscopic procedures. For example, the medical instruments can be configured for uroscopy, ureteroscopy, gastroscopy, bronchoscopy, or other endoscopic procedures. In some embodiments, the medical instruments can be configured for laparoscopic procedures or other types of medical procedures (e.g., open procedures).

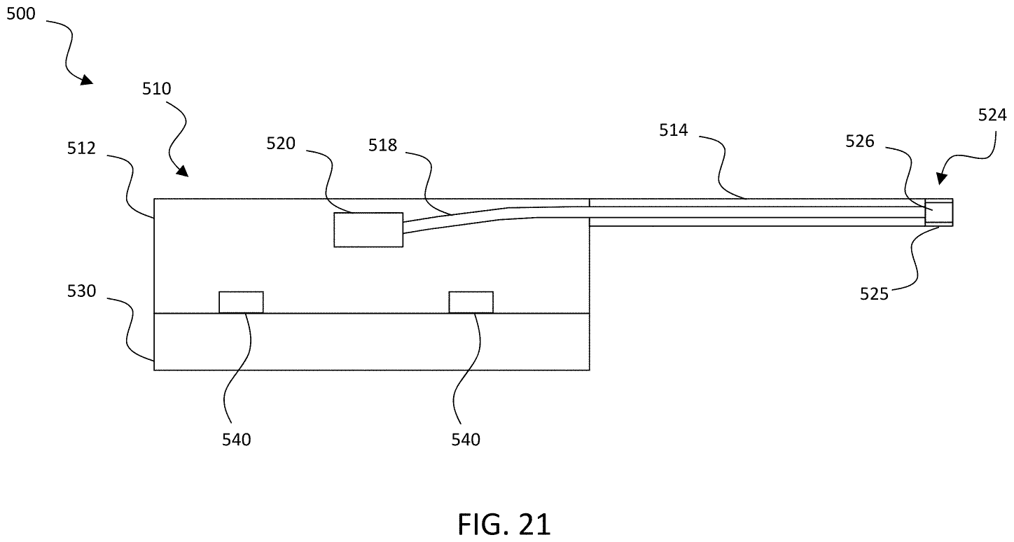

FIGS. 21-25 show various aspects of medical instruments that include illuminators. While FIG. 21 will be referenced here, it is noted that the subject matter of this disclosure is not limited to the example discussed. FIG. 21 shows an example robotic medical system 500. In some embodiments, the medical instrument 500 can include an instrument base 512 and a light source 520 disposed in the instrument base 512. The medical instrument 510 can include an elongate shaft 514 extending from the instrument base 512 and having a distal end with an optical fiber 518 extending along the elongate shaft 514. A wavelength conversion medium 526 can be disposed at the distal end of the elongate shaft 514. For example, the wavelength conversion medium 526 can include a luminescent material, such as a phosphor. The wavelength conversion medium 526 can be configured to emit light in response to receiving light from the optical fiber 518 and emit light having a new wavelength profile. In some embodiments, the optical fiber 518 can include a protective coating layer that is disposed around at least part of the optical fiber 518 but not around a distal end of the optical fiber 518. This can allow for better coupling of the optical fiber 518 to the wavelength conversion medium 526.

The wavelength conversion medium 526 can include an enclosure 525 extending from a distal section of the protective coating layer of the optical fiber 518 to at least a proximal section of the wavelength conversion medium 526. In some examples, for at least a portion of the enclosure 525, the enclosure 525 and the optical fiber 518 may be free of the protective coating layer there between. The wavelength conversion medium 526 can be disposed at least partially within an enclosure 525 that surrounds a distal section of the protective coating layer around the optical fiber 518. The enclosure 525 can extend from the distal section of the protective coating layer of the optical fiber 518 to the wavelength conversion medium 526. In some configurations, the optical fiber 518 is adhered and/or butt-coupled to the wavelength conversion medium 526.

The instrument base 512 can be coupled to a robotic arm 530 having a drive output (e.g., one or more of drive outputs 74, drive outputs 81) that is configured to articulate the elongate shaft 514 and steer the wavelength conversion medium 526, thereby directing light emitted from the luminescent material to a target anatomy. In some implementations, the medical instrument 510 can include a housing that is coupled to the robotic arm 530 such that the robotic arm 530 is configured to drive the elongate shaft axially through the housing.

The medical instruments can include one or more illuminators to help a user or practitioner (e.g., surgeon) to see the target anatomy. That light can be coupled into a proximal end of an optical fiber 518. The light may be coupled into the optical fiber 518 using one or more optical elements, such as a focusing element, a filtering element, a collimating element, a reflective element, and/or a polarizing element. Various light sources may be included, such as a white LED, a tungsten filament, a xenon light source, a laser, and/or some other broadband light source.

Monochromatic LEDs (e.g., green, blue, red, etc.) can have a bandgap semiconductor that emits light as current is passed through the semiconductor junction. According to some embodiments, a wavelength conversion medium such as a phosphor can be used to convert light from a monochromatic LED or other light source into white light. A phosphor is a type of wavelength conversion medium that can absorb blue photons from a blue light source, convert some of the photons into lower energies (e.g., green, yellow, orange, red), and elastically scatter others (e.g., some blue photons remain blue). Thus, white light is produced.

This configuration may be used in a flexible or rigid endoscope handle. This blue light source can be an LED or a laser diode. Laser diodes generally have higher brightness (e.g., flux per unit area) and can be more efficiently (e.g., greater than 70%) coupled into a multimode fiber than a white LED light source. The optical fiber 518 can, for example, have an outer diameter (OD) that is about 0.25 mm and can propagate the blue light to the endoscope tip (e.g., via total internal reflection (TIR)). The optical fiber 518 can have a polymer coating that protects the fiber and may enable it to withstand more rugged conditions than certain delicate (e.g., borosilicate) fibers. The optical fiber 518 would then emit the blue light at the tip directly into a phosphor (e.g., via a butt-coupling). The phosphor can be embedded in a silicone and/or or glass matrix. The matrix can be about 0.25 mm-0.3 mm thick and may form a round disk.

The configurations described herein can have a variety of advantages, depending on the configuration. For example, a blue laser diode may generate a fraction (e.g., about one quarter) of the waste heat in the instrument handle that may otherwise be generated with a white light LED but may have a comparable white light output at the tip of the endoscope. Additionally or alternatively, the blue light can be coupled into the fiber with a high efficiency (e.g., greater than 70%) than a coupling of white light from a white light LED (e.g., about 10% or less).

Additionally or alternatively, configurations described herein can include the use of certain optical fibers. The optical fiber 518 can be one that is hardened and that may be used by applications in telecommunications, data transport, and high-power laser amplifiers. Thus, in some configurations, the optical fiber 518 can withstand tortuous conditions better than more delicate optical fibers. The optical fiber 518 may be capable of delivering more than 100 W of power to the wavelength conversion medium 526 (e.g., phosphor) at the endoscope tip, which can allow for greater illumination intensities than some alternatives. In some implementations, at least 70 mW is used to achieve desired illumination intensity at the tip.

The wavelength conversion medium 526 can have a smaller footprint (e.g., about 0.25 mm thick) on the tip than other (e.g., borosilicate) optical fibers (e.g., about 0.5 mm thickness) and some LED-in-tip architectures (e.g., 0.5 mm×0.9 mm). The phosphor can function as a diffuser to more uniformly distribute the incoupled light (e.g., blue). Thus, the generated light can be emitted at a wide angle. In some embodiments, the light has an approximately uniform intensity distribution across the wide angle. The wavelength conversion medium 526 can generate a fraction of the heat that certain configurations (e.g., some LED-in-tip architectures) produce since the heat generated may be generally associated with the quantum defect of converting higher energy light to lower energies.

How much light can be produced with a white light LED can be influenced by (e.g., limited) the heat generated in the proximity of illumination from the electro-optical conversion. In certain configurations, the wavelength conversion medium 526 (e.g., phosphor) can be separated from the light source 520 (e.g., blue LED), which can mitigate the waste heat dissipation into the wavelength conversion medium.

As noted generally above, incoupled light can be guided to a distal end of the optical fiber 518. At the distal end of the optical fiber 518, a wavelength conversion material can convert the incoupled light to light having a new or second wavelength profile (e.g., white). This process may decouple aspects of the generation of white light. For example, white light may be generated from a blue (˜450 nm) light source. The blue light excites a phosphor embedded in a matrix, which has a fluorescence/phosphorescence that complements the original blue spectrum with red and green counterparts. In some configurations, the illuminators described herein may produce the light at a proximal end and convert the light at a distal end, rather than having the light production and conversion occur at the same location. The illumination can be applied in the context of medical instruments, such as robotic medical instruments and/or their related systems.

The medical instruments may include illuminators to project light onto patient anatomy. Various illumination architectures may be used. For example, one or more LEDs or laser diodes may be placed in the handle of a flexible or rigid elongate medical instrument. Additionally or alternatively, a light source can be included in a tower or console remote from the medical instrument. In either configuration, a light guide such as an optical fiber can be used to convey the light to an illumination component at a distal tip of the medical instrument where illumination of the target is desired. The illumination component can include a wavelength conversion medium to convert the received light into a desired spectrum (e.g., white light). Additionally or alternatively, the illumination component can be configured to diffuse or scatter the received light to widen the illumination angle or illumination cone to facilitate directing the light towards the desired illumination target. Additionally or alternatively, the illumination component can include a light emissive or luminescent material that emits light towards the target in response to receiving the light from the target.

Architectures described herein may include a light source 520 that is decoupled from a wavelength conversion medium 526, which converts light to different wavelength profiles for one or more imaging techniques. As noted above, for example, white light may be used as the resulting wavelength profile. However, other wavelength profiles may be used for other imaging techniques. For example, the configurations described herein can be used for fluorescence imaging (e.g., using contrast agents such as ICG or 5-ALA), auto fluorescence imaging (AFI), multi-band imaging (MBI), narrow band imaging (NBI), and/or nonlinear imaging (e.g., light conversion at the tissue). In some implementations, to change among various wavelength profiles, only the light source 520 (e.g., laser diode, LED) is changed for the same wavelength conversion medium.

With reference once again to FIG. 21 and as noted above, provided is the example robotic medical system 500, according to some embodiments, that can include the medical instrument 510 and an instrument drive mechanism of the robotic arm 530. The instrument drive mechanism of the robotic arm 530 can correspond to any instrument drive mechanism described herein, such as the instrument drive mechanism 146A/146B described above with respect to FIG. 14, whereas the robotic arm 530 can correspond to any robotic arm described herein, such as the robotic arm 76 described above with respect to FIG. 16, and/or the robotic arm 82 described above with respect to FIG. 17. The medical instrument 510 can include the instrument base 512, the elongate shaft 514 coupled to a distal end of the instrument base 512, the light source 520 coupled to a proximal end of the optical fiber 518, and the distal illumination component 524 coupled to the optical fiber 518.

In particular, it is noted that the light source 520 can be disposed inside or outside the instrument base 512. As shown in FIG. 21, the light source 520 is disposed inside the instrument base 512. The light source 520 can be optically coupled to the optical fiber 518 such that light generated by the light source 520 is coupled into the optical fiber 518. Various coupling efficiencies (e.g., optical output of the light source 520 compared to the light incoupled into the optical fiber 518) are possible.

The optical fiber 518 can be disposed within the elongate shaft 514. Other elements of the robotic medical system 500 may be disposed within the elongate shaft 514, as described herein (e.g., a working channel, an elongate instrument, etc.). The optical fiber 518 can have a protective coating disposed around a fiber core. The fiber core can be configured to transmit incoupled light to a distal end of the optical fiber 518 via total internal reflection (TIR). The elongate shaft 514 can be flexible (e.g., in endoscopic or endoluminal procedures) or rigid (e.g., in laparoscopic procedures).

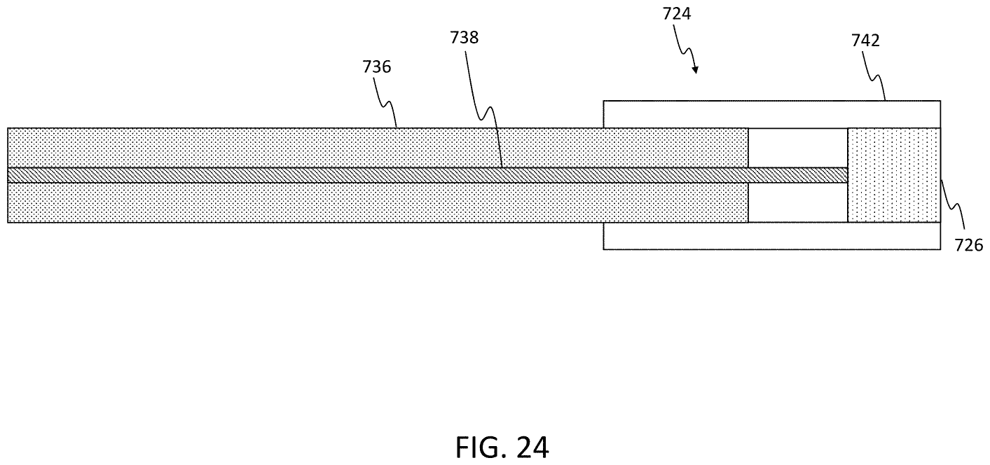

The distal illumination component 524 can disposed at a distal end of the optical fiber 518 or a portion thereof (e.g., the fiber core, as explained herein). The distal illumination component 524 may include an enclosure 525 disposed around a wavelength conversion medium 526. The distal illumination component 524 may share one or more features with the distal illumination component 724 discussed below with respect to FIG. 24.

The wavelength conversion medium 526 can be configured to convert the incoupled light having a first wavelength profile (e.g., blue) to a second wavelength profile (e.g., white). Various wavelength profiles are possible. For example, the first wavelength profile can be within the blue spectrum (e.g., about 405 nm to about 500 nm). The first wavelength profile can be centered around a wavelength disclosed herein, such as about 410 nm, about 420 nm, about 430 nm, about 440 nm, about 450 nm, about 460 nm, about 470 nm, about 480 nm, about 490 nm, about 500 nm, any wavelength value therebetween or falling within a range having endpoints of values therein. For example, in some embodiments, the first wavelength profile centers around 450 nm. While some colors have wavelength ranges spanning 70 nm or more, narrower wavelength ranges are possible within the visible spectrum, such as wavelength ranges of about 10 nm, about 20 nm, or about 30 nm. In some embodiments, light outside the visible spectrum (e.g., ultraviolet light) may be used as the first wavelength profile.

The robotic arm 530 can be coupled to the medical instrument 510 (e.g., at the instrument base 512, as shown in FIG. 21). The robotic arm 530 can include one or more robotic drive outputs (not shown). The robotic arm 530 may be directly coupled to the instrument base 512 via the one or more robotic drive outputs, which may correspond to respective one or more robotic drive inputs 540. The one or more robotic drive inputs 540 can drive actuators for actuating the elongate shaft 514 and/or the optical fiber 518. For example, the robotic drive inputs 540 may translate, articulate (e.g., as described herein), and/or rotate the optical fiber 518. Due to the coupling of the optical fiber 518 to the distal illumination component 524, the distal illumination component 524 may thereby be indirectly or directly actuated by the robotic drive inputs 540. Additionally or alternatively, the robotic drive inputs 540 may be configured to actuate the elongate shaft 514 itself. For example, the robotic drive inputs 540 may be configured to translate the elongate shaft 514 axially within the instrument base 512. The robotic arm 530 may correspond to robotic arms described herein. Additionally or alternatively, the robotic drive inputs 540 can correspond to any of the robotic drive inputs disclosed herein. Other elements of the robotic medical system 500 can correspond to the functionality of elements described herein as well. These may not be fully disclosed here for brevity and to avoid unnecessary duplication of discussion above with, for example, reference to FIGS. 1-20.

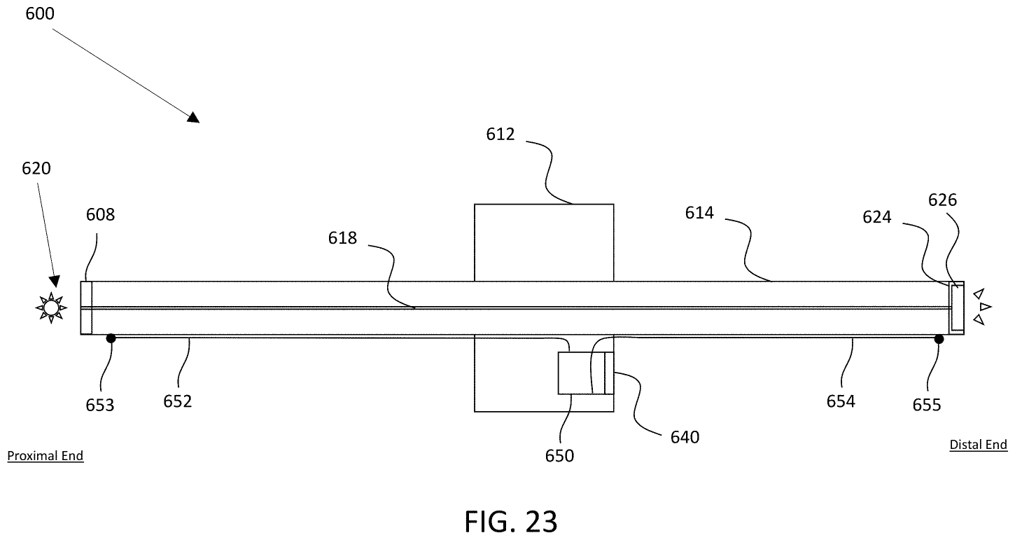

The light source 520 can include an LED, a laser diode, a xenon light source, a filament light source, and/or any other light source. The optical fiber 518 can be a multimode fiber. The optical fiber 518 can have an outer diameter (OD) that is about 0.1 mm, about 0.15 mm, about 0.2 mm, about 0.25 mm, about 0.3 mm, about 0.35 mm, about 0.4 mm, about 0.45 mm, about 0.5 mm, about 0.6 mm, about 0.7 mm, about 0.8 mm, about 0.9 mm, about 1 mm, have any OD value between, or have an OD that falls within any range having endpoints therein. An optical coupler (not shown) may be configured to couple the light from the light source 520 into the optical fiber 518. The optical coupler may have one or more features shared with the optical coupler 608 disclosed below with reference to FIG. 23.

The optical fiber 518 can have a protective coating that protects the fiber core (not shown in FIG. 21). The coating can be a flexible material, such as a polymer. The protective coating can enable it to withstand damage from other elements inside the instrument base 512, the elongate shaft 514, and/or any other elements of the robotic medical system 500.

The light source 520 may generate less waste heat in the instrument base 512 than would be generated with a white light LED therein. Additionally or alternatively, the light from the light source 520 can be coupled into the optical fiber 518 with greater efficiency than would be coupled from a white light LED.

The optical fiber 518 can be capable of delivering a high power to the wavelength conversion medium 526. The power delivered can be between about 5 milliwatts and 10 W, any power value there between, or fall within any range having endpoints therein.

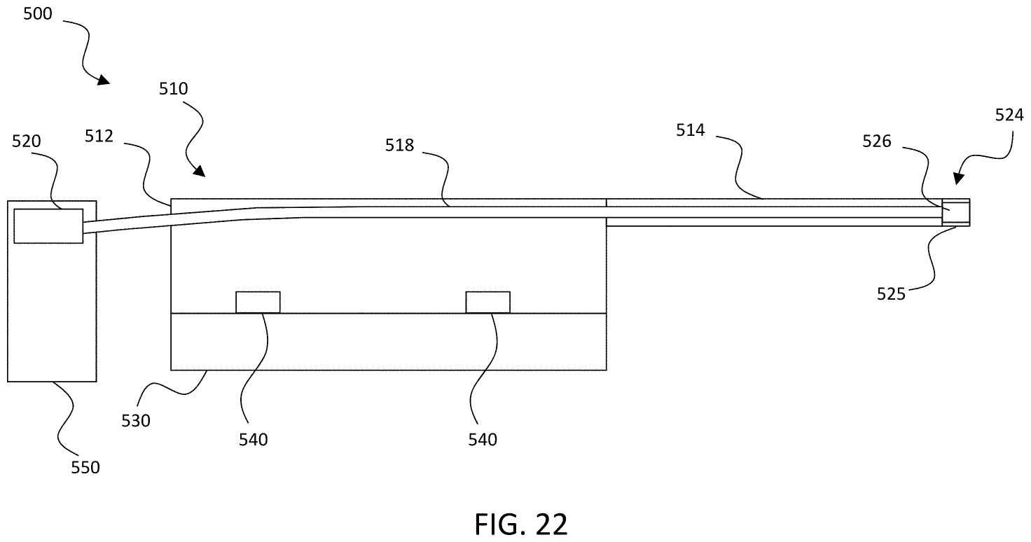

FIG. 22 shows an example configuration of a robotic medical system 500 where the light source 520 is disposed outside of the medical instrument 510. As shown, the light source 520 can be disposed inside a tower 550. The tower 550 may be a tower for controlling various aspects of the robotic medical system 500. The tower 550 may be the tower 30 described above with respect to FIG. 1.

The optical fiber 518 may begin inside the tower 550 and may extend outside the tower 550 as shown. Additionally or alternatively, the optical fiber 518 may enter the instrument base 512 (e.g., from a proximal end of the instrument base 512) via a fiber aperture. The tower 550 may be movable with respect to the medical instrument 510. Thus, in some configurations, the optical fiber 518 is configured to extend from the tower 550 to the distal illumination component 524. In some configurations, two or more optical fibers may be used that are connected to one another via an adaptor configured to couple light from one optical fiber to another.

FIG. 23 shows an example robotic medical system 600 where an elongate shaft 614 is configured to translate axially through an instrument base 612. The robotic medical system 600 can include the instrument base 612, the elongate shaft 614, a robotic actuator 650, and a light source 620. As with the light source 520 in FIGS. 21-22, the light source 620 may be configured to emit light at a first wavelength profile and couple light into an optical fiber 618. The light can then be incident on a wavelength conversion medium 626 of a distal illumination component 624. The distal illumination component 624 may share one or more features with the distal illumination component 524 discussed above with reference to FIGS. 21-22 and/or with the distal illumination component 724 discussed below with respect to FIG. 24.

The light source 620 may be disposed outside the instrument base 612 and outside the elongate shaft 614 in some configurations. In some implementations, the light source 620 may be positioned as in the light source 520 in either FIG. 21 or FIG. 22. Because in FIG. 23 the elongate shaft 614 is configured to translate relative to the instrument base 612, it may be advantageous to position the light source 620 outside the instrument base 612. This positioning may allow the light source to be coupled into a fiber that translates with the elongate shaft relative to the instrument base, without a need for tightly coiling the optical fiber 618.

The elongate shaft 614 can be configured to translate along an axis extending in distal and proximal directions, as shown. The elongate shaft 614 can be rigid. Such a rigid elongate shaft 614 may be used, for example, in laparoscopy. As with other configurations disclosed herein, the elongate shaft 614 can include a working channel, one or more instruments, and/or other elements not shown in FIG. 23.

The elongate shaft 614 may be actuated (e.g., translated) using one or more pull wires. As shown, for example, the robotic medical system 600 can include a first pull wire 652 and a second pull wire 654. The first pull wire 652 may be coupled (e.g., attached) to a proximal portion (e.g., a proximal end) of the elongate shaft 614 at a first pull wire coupling or termination point 653. Additionally or alternatively, the second pull wire 654 may be coupled to a distal portion (e.g., a distal end) of the elongate shaft 614 at a second pull wire coupling or termination point 655. The robotic actuator 650 can include one or more robotic drive inputs 640. The robotic drive inputs 640 can be configured to receive actuation from corresponding robotic drive outputs (not shown) in the robotic medical system 600. The robotic drive inputs 640 may have functionality of other robotic drive inputs disclosed above.

The light source 620 may be configured to couple the light into the optical fiber 618 via an optical coupler 608. The optical coupler 608 can include one or more optical elements. For example, the optical coupler 608 can include a reflective optical element (e.g., mirror), a refractive optical element (e.g., a focusing lens, a collimating lens), a filtering optical element (e.g., polarizer), and/or other optical element. In some examples, one or more optical elements of the optical coupler 608 may be disposed at least partially proximally of the light source 620, such as a reflective optical element. The reflective optical element may include a collimating mirror. Other variants are possible.

FIG. 24 shows an example configuration of a distal illumination component 724 that may be used with any of the robotic medical systems discussed herein. An optical fiber 738 (which may correspond to the optical fiber 518 and/or the optical fiber 618 discussed above) can comprise a core and a cladding. A protective coating layer 736 can surround at least a portion of the optical fiber 738. The protective coating layer 736 can be flexible and/or resilient. The protective coating layer 736 may include a polymer coating. The protective coating layer 736 can include acrylate in some configurations. The optical fiber 738 may include a transparent material in its core. The fiber core may include silica and/or plastic. The distal illumination component 724 may include a wavelength conversion medium 726 and an enclosure 742. The enclosure 742 may include a glass or heat shrink material such as polytetrafluoroethylene (PTFE). The enclosure 742 may be shaped as a tubing surrounding at least a portion of the wavelength conversion medium 726 and a distal end of the fiber 738.

The distal end of the optical fiber 738 may be directly coupled (e.g., adhered, attached) to the wavelength conversion medium 726. The wavelength conversion medium 726 may correspond to the wavelength conversion medium 526 and/or wavelength conversion medium 626 discussed above with respect to FIGS. 21-23. The protective coating layer 736 may terminate proximal of the wavelength conversion medium 726. This may allow the optical fiber 738 to have a better coupling with the wavelength conversion medium 726. Such a recessed protective coating layer 736 may reduce light leakage, heat waste, and/or long-term damage to the optical fiber 738 and/or to the wavelength conversion medium 726. The optical fiber 738 may be butt-coupled to the wavelength conversion medium 726 in some configurations. The optical fiber 738 can be configured for multimode or single mode.

The enclosure 742 may surround a distal end of the optical fiber 738. For example, the enclosure 742 may be coupled to a distal end of the protective coating layer 736 and/or may form an enclosure of a distal end of the optical fiber 738. In this way, the enclosure 742 may serve as a protective element of the optical fiber 738. The enclosure 742 can extend from the protective coating layer 736 to the wavelength conversion medium 726. The enclosure 742 may surround the wavelength conversion medium 726 as well. The enclosure 742 can have an inner diameter (ID) that is equal to or larger than the OD of the wavelength conversion medium 726. According to some embodiments, the protective coating layer 736 can have a diameter of between about 100 and about 400 microns, and in some configurations, about 250 microns. The optical fiber 738, which can have a core with a diameter of between about 80 microns to about 200 microns, and in some configurations is about 125 microns.

In some embodiments, the enclosure 742 may include a glass. In some embodiments, the enclosure 742 may include a polymer, such as a polyimide. The enclosure 742 may be heat shrunk around the optical fiber 738 and/or the wavelength conversion medium 726.

The wavelength conversion medium 726 can include a phosphor. In some configurations, the phosphor is embedded in a silicone and/or or glass matrix. Additionally or alternatively, the phosphor may be embedded in a polymer matrix. The wavelength conversion medium 726 can include barium borate (BBO), such as BBO crystals. Additionally or alternatively, the wavelength conversion medium 726 can include lithium and/or nobate, such as in periodically poled lithium nobate (PPLN). Thus, various first and second wavelength profiles may be possible.

According to some embodiments, the wavelength conversion medium 726 can be about 0.1 mm to about 0.7 mm thick. Thickness may be measured axially. In some configurations, the wavelength conversion medium 726 has a thickness of about 0.25 mm. The wavelength conversion medium 726 may form a cylinder (e.g., a round disk). The wavelength conversion medium 726 can have an OD of between about 0.2 mm and about 1 mm, and in some embodiments is about 0.5 mm. The phosphor can function as a diffuser to more uniformly distribute the incoupled light (e.g., blue light). Thus, the generated light can be emitted in all directions approximately equally.

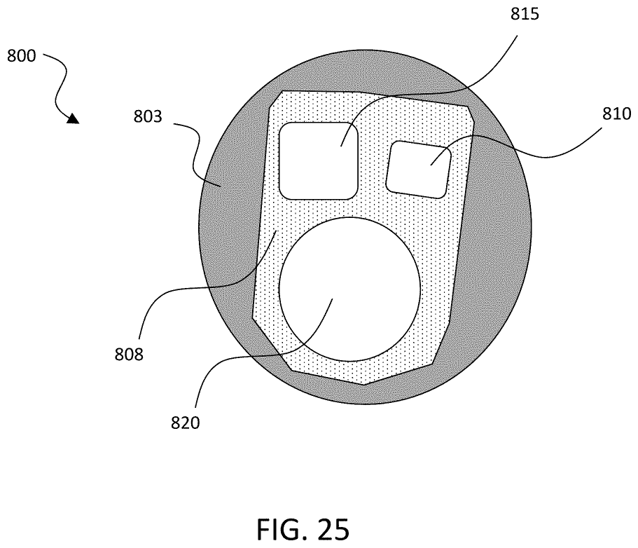

With reference to FIG. 25, provided is an end view of a distal end or face 808 of an elongate shaft 803 of an example medical instrument 800. The medical instrument 800 may be representative of the elongate shaft 514 of FIGS. 21-22. The medical instrument 800 may be representative of any medical instrument described throughout the disclosure, such as the endoscope 13 of FIG. 1, the ureteroscope 32 of FIG. 3, the laparoscope 59 of FIG. 9, etc. A distal portion of the elongate shaft 803 can be rounded or tapered toward the distal end 808 of the medical instrument 800 to provide an atraumatic surface.

The distal end 808 may have one or more apertures or ports as shown in FIG. 25A. For example, the distal end 808 can have three apertures. A first aperture may correspond to the distal end of a working channel 820. The working channel 820 may be configured to allow for surgical instruments (e.g., biopsy needles, cytology brushes, forceps, etc.) to pass therethrough to allow access to the area near the instrument tip (i.e., the distal end 808 in the present example).

A second aperture may include a distal illumination component 810 that is disposed therein to provide illumination therethrough. The distal illumination component 810 can be configured to illuminate a target within the patient, and can have a wavelength conversion medium and/or luminescent material to provide desired wavelengths of light to the target. For example, the distal illumination component 810 can correspond to the distal illumination component 724 (FIG. 24). The wavelengths can be any suitable wavelength, for example, visible spectrum light, ultraviolet light, infrared light, x-ray (e.g., for fluoroscopy), to name a few examples. In some embodiments, the distal illumination component 810 can include one or more LEDs, lasers, and/or wavelength conversion mediums located at the distal end of the instrument 800. In some embodiments, the illuminator 810 can include one or more fiber optic fibers extending through the length of the endoscope to transmit light through the distal end from a remote light source (e.g., in an instrument base, in a separate tower, etc.). A shown, a single illuminator 810 can be included, but in other embodiments (not shown) multiple illuminators can be included at the distal end 808.

A third aperture may include an imaging device 815 therein. The imaging device 815 can include an image sensor held within the aperture at the distal end 808 and configured to capture images of the illuminated target within the patient. According to some embodiments, the imaging device 815 may include any photosensitive substrate or structure configured to convert energy representing received light into electric signals, for example, a charge-coupled device (CCD) or complementary metal-oxide semiconductor (CMOS) image sensor. According to some embodiments, the imaging device 815 can include one or more optical fibers configured to transmit light representing an image from the distal end 800 of the endoscope to an eyepiece and/or image sensor located remote from the distal end. According to some embodiments, the imaging device 815 can include one or more lenses and/or wavelength pass or cutoff filters as useful for various optical designs. The light emitted from the illuminator 810 can allow the imaging device 815 to capture images of a patient. These images can then be transmitted as individual frames or series of successive frames (e.g., a video) to a computer system.

According some embodiments, EM sensors such as EM coils may be located near the distal end of the instrument 800 may be used with an EM tracking system to detect the position and orientation of the distal end of the instrument 800. In some embodiments, the coils may be angled to provide sensitivity to EM fields along different axes, giving the disclosed navigational systems the ability to measure a full 6 degrees of freedom (DoF): three positional DoF and three angular DoF. In other embodiments, only a single coil may be disposed on or within the distal end with its axis oriented along the instrument shaft. Alternatively or additionally, other types of position sensors may be employed.

The apertures at the distal end of the instrument 800 may provide physical and/or optical access for working channel instruments, illumination, and image capture via the various devices that are disposed within or extendable through their respective ports. According to some embodiments, the EM sensors may be able to function without physical or optical access through the distal end of the instrument to function and may be held within an enclosed part of tip of the elongate shaft, proximal to the aperture openings at the distal end.

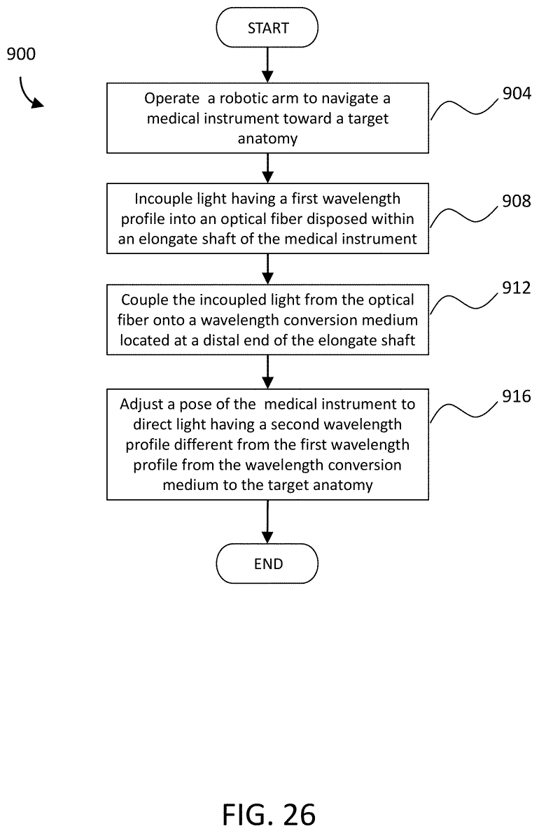

FIG. 26 is a flowchart illustrating an example method 900 for illuminating a target using a robotic medical system described herein with respect to FIGS. 1-25, in accordance with aspects of this disclosure. For example, the robotic medical system can include one or more steps represented by blocks performed by processor(s) and/or other component(s) of a robotic system (e.g., robotic medical system) or associated system(s). The method 900 begins at block 904, wherein the robotic medical system can include operating a robotic arm to navigate a medical instrument toward the target anatomy. For example, robotic drive inputs (e.g., robotic drive inputs 540 of FIGS. 21-22) may be configured to translate an elongate shaft of the medical instrument axially within the instrument base of the medical instrument. The robotic arm can drive actuators for actuating the elongate shaft and/or an optical fiber of the system.

At block 908, the robotic medical system can include incoupling light having a first wavelength profile into an optical fiber disposed within an elongate shaft of the medical instrument. The system can transmit incoupled light to a distal end of an optical fiber via total internal reflection (TIR).

At block 912, the robotic medical system can include coupling the incoupled light from the optical fiber onto a wavelength conversion medium located at a distal end of the elongate shaft. The wavelength conversion medium (e.g., the wavelength conversion medium 526 of FIGS. 21-22) can convert the incoupled light having a first wavelength profile (e.g., blue) to a second wavelength profile (e.g., white). Various features of the wavelength conversion medium 526 can apply to the wavelength conversion medium of block 912.

At block 916, the robotic medical system can include adjusting a pose of the medical instrument to direct light having a second wavelength profile different from the first wavelength profile from the wavelength conversion medium to the target anatomy. Adjusting the post of the medical instrument may include articulating, translating, and/or rotating the elongate shaft in one or more degrees of freedom described herein. In some configurations, the robotic medical system can include translating the elongate shaft along an axis defined by the elongate shaft. The method 900 can end after block 916, though the method 900 can include additional steps not shown in FIG. 26.

### 3. Implementing Systems and Terminology

Implementations disclosed herein provide systems, methods and apparatus related to medical instruments having one or more illuminators.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The phrases referencing specific computer-implemented processes and functions described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A robotic medical system, comprising:
a medical instrument comprising:
an elongate shaft having a distal end,
an optical fiber extending along the elongate shaft, and
a wavelength conversion medium disposed at the distal end of the elongate shaft, the wavelength conversion medium being configured to receive light having a first wavelength profile from the optical fiber and emit light having a second wavelength profile different from the first wavelength profile; and
a robotic arm configured to manipulate the medical instrument, wherein the robotic arm is articulable to position the medical instrument with respect to a patient, and wherein the robotic arm is configured to actuate the elongate shaft to move the wavelength conversion medium within the patient.

2. The system of claim 1, wherein a distal end of the optical fiber is coupled to the wavelength conversion medium.

3. The system of claim 1, further comprising a protective coating layer disposed around at least a portion of the optical fiber, wherein the protective coating layer terminates proximally of a coupling between the optical fiber and the wavelength conversion medium.

4. The system of claim 3, wherein the wavelength conversion medium is disposed at least partially within an enclosure that surrounds a distal section of the protective coating layer, the enclosure extending from the distal section of the protective coating layer of the optical fiber to the wavelength conversion medium.

5. The system of claim 1, wherein the medical instrument further comprises a housing coupled to the robotic arm, wherein the robotic arm is configured to drive the elongate shaft axially through the housing.

6. The system of claim 1, wherein the wavelength conversion medium comprises a phosphor.

7. The system of claim 1, wherein the optical fiber is configured to receive, at a proximal end thereof, light having the first wavelength profile and to transmit the received light via total internal reflection to the wavelength conversion medium.

8. The system of claim 1, further comprising a light source coupled to the optical fiber and configured to emit light at a frequency between about 380 nm to about 500 nm and wherein the wavelength conversion medium is configured to emit light including wavelengths greater than about 500 nm and less than about 380 nm in response to light having the first wavelength profile being incident thereon.

9. The system of claim 1, further comprising a light source disposed outside the elongate shaft of the medical instrument, wherein the light source is configured to emit light having the first wavelength profile into the optical fiber.

10. The system of claim 1, wherein the elongate shaft comprises a first aperture at a distal end of the elongate shaft, the first aperture configured to receive the wavelength conversion medium therein.

11. The system of claim 10, wherein the elongate shaft further comprises a second aperture configured to receive an image sensor therein.

12. The system of claim 11, wherein the elongate shaft further comprises a third aperture configured to receive a surgical instrument there through.

13. A medical instrument, comprising:
an instrument base;
an elongate shaft extending from the instrument base, the elongate shaft having a distal end;
an optical fiber extending along the elongate shaft;
a light source disposed in the instrument base, the light source being configured to emit light into the optical fiber; and
a luminescent material disposed at the distal end of the elongate shaft, the luminescent material being configured to emit light in response to receiving light from the optical fiber.

14. The instrument of claim 13, wherein the optical fiber comprises a protective coating layer disposed around at least part of the optical fiber but not around a distal end of the optical fiber.

15. The instrument of claim 14, further comprising an enclosure around the luminescent material, the enclosure extending from a distal section of the protective coating layer to at least a proximal section of the luminescent material, wherein, for at least a portion of the enclosure, the enclosure and the optical fiber are free of the protective coating layer there between.

16. The instrument of claim 13, wherein the optical fiber is butt-coupled to the luminescent material.

17. The instrument of claim 13, wherein the instrument base is configured to be coupled to a robotic arm having a drive output configured to articulate the elongate shaft and steer the luminescent material, thereby directing light emitted from the luminescent material to a target anatomy.

18. A method operable by a robotic medical system to illuminate a target anatomy, the method comprising:
operating a robotic arm to navigate a medical instrument toward the target anatomy;
incoupling light having a first wavelength profile into an optical fiber disposed within an elongate shaft of the medical instrument;
coupling the incoupled light from the optical fiber onto a wavelength conversion medium located at a distal end of the elongate shaft; and
adjusting a pose of the medical instrument to direct light having a second wavelength profile different from the first wavelength profile from the wavelength conversion medium to the target anatomy.

19. The method of claim 18, wherein adjusting the pose of the medical instrument comprises translating the elongate shaft along a longitudinal axis of the elongate shaft.
